(19) United States
(12) Patent Application Publication
(10) Pub. No.: US 2017/0290727 A1
(43) Pub. Date: Oct. 12, 2017

(54) CERVICAL TRACTION DEVICE AND METHOD OF USING SAME

(71) Applicant: Elton Devin Savage, Boeblingen (DE)

(72) Inventor: Elton Devin Savage, Boeblingen (DE)

(21) Appl. No.: 15/095,307

(22) Filed: Apr. 11, 2016

(51) Int. Cl. A61H 1/02; A61F 5/042

(52) U.S. Cl. CPC A61H 1/0218; A61F 5/042; A61H 2201/1607; A61H 2201/1664

(57) ABSTRACT

A cervical traction device is provided. A frame presents an anterior support under the maxilla of a user, while a posterior support is applied to the lower portion of the occiput of the user. Thus, pressure is avoided on the potentially unstable mandible, and the lower and upper teeth are not compressed against one another. A series of suspension points on the frame allows the angle at which traction may be applied to the cervical vertebrae to be varied.

### STATEMENT REGARDING FEDERALLY SPONSORED RESEARCH OR DEVELOPMENT

Not applicable.

### TECHNICAL FIELD

The present disclosure relates generally to a device for applying cervical traction to a human user, and to a method of using the same.

### BACKGROUND OF THE INVENTION

Traction is a therapeutic method to relieve pain by stretching and realigning the spine. Placing a stretch on the spine separates the vertebrae and helps to relieve direct nerve pressure and stress on the vertebral discs. Cervical traction is a common nonsurgical treatment for a herniated disc, and other conditions of the spine and musculature in the neck that relieves pain by opening up the cervical foramen to reduce pressure on compressed nerve roots exiting the spinal canal. Traction can either be applied manually or by spinal traction devices.

The history of traction as a medical instrumentality is lengthy and well-known. The Russian neurologist Osip Mochutkovsky described suspension as a method for the treatment of tabes dorsalis in an article published in the Russian magazine “Vratch” in 1883. His works became known in Europe when J.M. Charcot paid attention to it and published a special short monograph on this subject in 1889. This work was translated into English (1889) and Russian (1890) and the method became popular in the treatment of tabes dorsalis and other neurological diseases. The Russian neurologist V.M. Bekhterev proposed the combination of body suspension with cervical traction (1893). Some years later Gilles de la Tourette promoted the use of spinal traction in his neurological clinic (1897).

Various methods of treating pain in the cervical region resulting from structural or other irregularities of the spine are known in the art. For example, one method used for the treatment of pain in the neck region involves cervical traction, in which at least a portion of the cervical region of the spinal column is stretched to achieve axial distraction of the spine and reduce axial stresses on the discs and facet joints. Such cervical traction can be achieved through the use of conventional over-the-door static traction, where a person sits and wears a head halter that is attached to a weight via a rope and pulleys, and the pulleys being mounted to a bracket attached to a top edge of a closed door. Traction of the cervical region of the spine may also be achieved by placing a patient in a chair or on a semi-supine treatment/examining table, fitting a portion of the cervical spine with a traction strap, and exerting a static traction load to pull the strap and thereby transversely stretch the spine. In these methods, the static traction force applied to the body attempts to induce an extension of the cervical region of the spine that is maintained at an intensity and for a duration sufficient to induce muscle and ligament changes, thereby assisting the spine into a more proper lateral curvature.

A problem with conventional static, extension traction methods is that they often do not achieve a proper effect on the spine within an acceptable time frame. For example, because the traction force applied during treatment is limited by the patient's tolerance level, proper treatment may require many repeated traction sessions in order to achieve the desired treatment outcome. This is a problem because the scheduling and coordinating of multiple sessions can be inconvenient for many patients, and also patient compliance with the treatment program may decrease when numerous sessions are involved. Also, the patients may continue to suffer from unacceptable levels of pain until the multiple required traction sessions have been completed. Yet another problem with conventional static extension traction methods is that most patients experience discomfort during prolonged static traction sessions due to the constant and unchanging tension exerted in the spine.

Application of cervical traction is also impaired by the difficulty of making suitable fixation to the head of a human user, which is a more or less rounded object. By far the most common means of attaching cervical traction to a user involves a first strap around the occipital base of the skull, and a second strap around the mandible of the subject, as seen in U.S. Pat. No. 4,220,147. This has many disadvantages in practice. The temporomandibular joint (TMJ) is a relatively freely moving and delicate one, and therefore fixation below the mandible is inherently unstable and uncomfortable. Also, traction on the mandible tends to compress the jaw and teeth, with attendant discomfort and even danger to the patient. Accordingly, there remains a need for a safe and efficient means of treating patients' abnormalities of the spine in the cervical region of the spine, without excessive pain or discomfort.

### SUMMARY OF THE INVENTION

These challenges, among others, are solved by various embodiments of the present invention. Illustrative examples of various embodiments of the invention, all provided by way of example and not limitation, are described.

A cervical traction device may include a frame and two non-traumatic and releasable skull attachments. A first skull attachment may be attached to the frame where it would be releasably attachable to a user at an anterior portion of a head on an inferior aspect of a maxilla of the user. Thus upward force would be exerted on the maxilla, and not the mandible as is common in the prior art. There may be a second user attachment attached to the frame where it would be releasably attachable to the user at a posterior portion of the head on an inferior aspect of an occiput of the user. Thus, the total upwards traction force could be distributed between the back of the head and the maxilla.

The skull attachments may be fixed to the frame, or they may be releasably attached, and may exist as a plurality of attachments to vary the positon of the user attachments relative to the frame. A plurality of suspension points (110) may help attach a device external to the frame (100) such as a source of cervical traction. The plurality of suspension points (110) may be arranged in a front-to-back manner, thereby allowing the angle at which traction is applied to the cervical vertebrae to be adjusted and varied.

The first reversible skull attachment may be a maxillary tray releasably supporting the maxilla of the user, and may fit over the teeth. For edentulous users or those with few teeth, the tray may be formed to fit directly against the soft palate, over the upper gum. The tray may have a tooth guard on its' superior surface and the tooth guard may molded to an individual maxillary dentition of a unique user, or similarly, may be molded to the gums and/or soft palate of a user with few or no teeth.

The second reversible skull attachment (300) may be an occipital strap (310), adjustable both in length and with regard to its position of attachment to the frame (100). Overall, in some embodiments, the device is intended so that the frame, and hence any traction force applied, does not contact a mandible of a user while in use. However, one skilled in the art will understand that incidental contact with the mandible, or even some intentional level of force applied to the mandible, will be possible in some other embodiments.

The frame may be formed of two or more reversibly joined sections that may be disassembled. The frame may also be fabricated in a geometric shape that is substantially planar. Such a planar shape would minimize the storage space required by the frame and make it more efficient for travel and transport.

### BRIEF DESCRIPTION OF THE ILLUSTRATIONS

Without limiting the scope of the cervical traction device as disclosed herein and referring now to the drawings and figures:

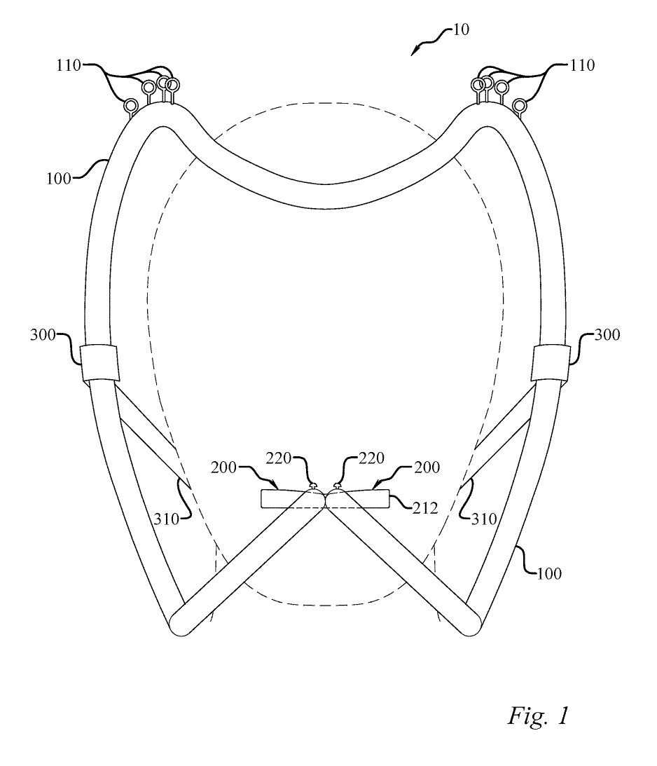

FIG. 1 shows a front elevation view of an embodiment of the present invention, with the head of a human user using the device shown in broken line;

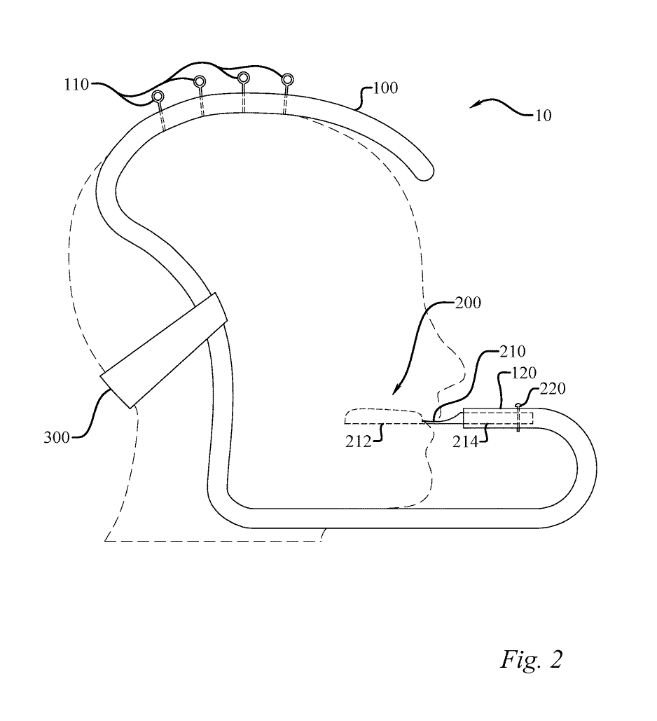

FIG. 2 shows a side view of the embodiment of FIG. 1, with the head of a human user using the device shown in broken line;

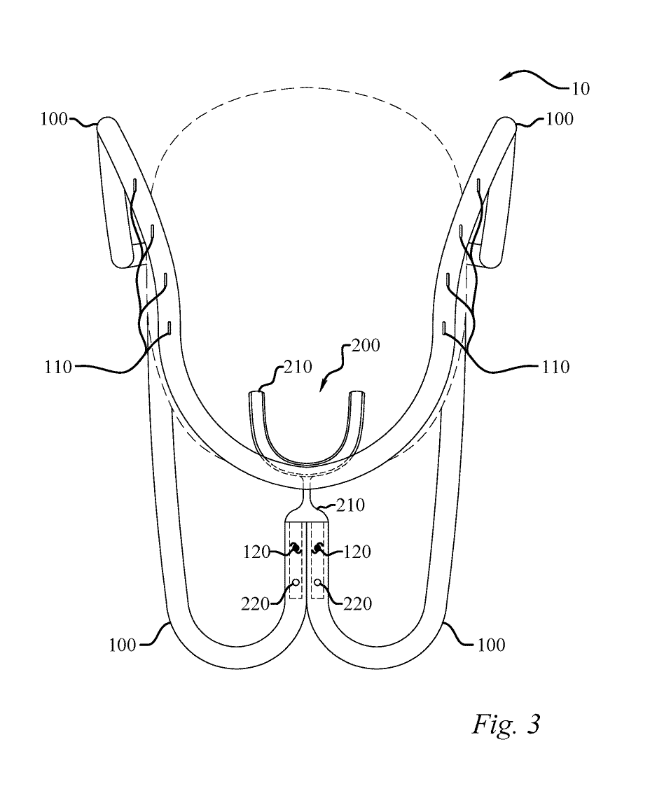

FIG. 3 shows a top view of the embodiment of FIG. 1, with the head of a human user using the device shown in broken line;

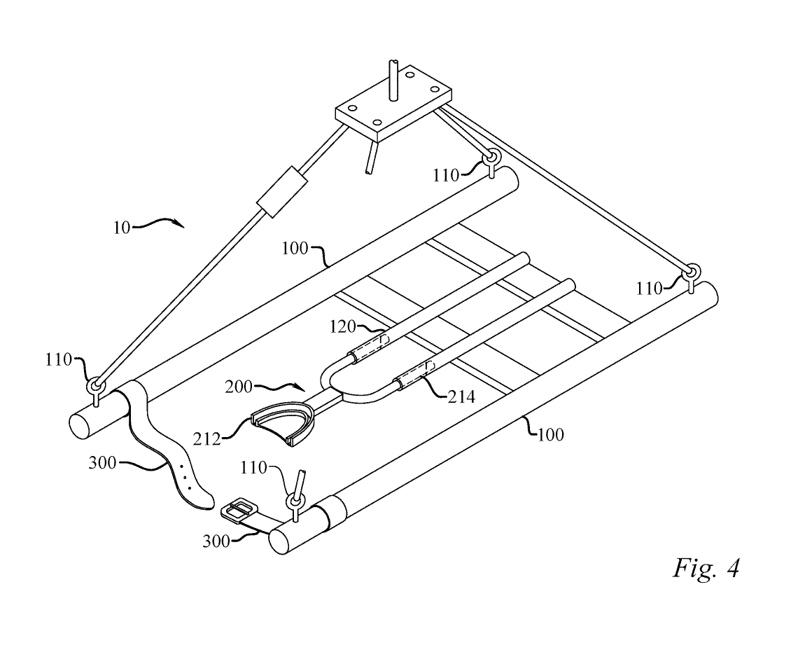

FIG. 4 shows a perspective view of another embodiment of the present invention, with the head of a human user using the device shown in broken line; and

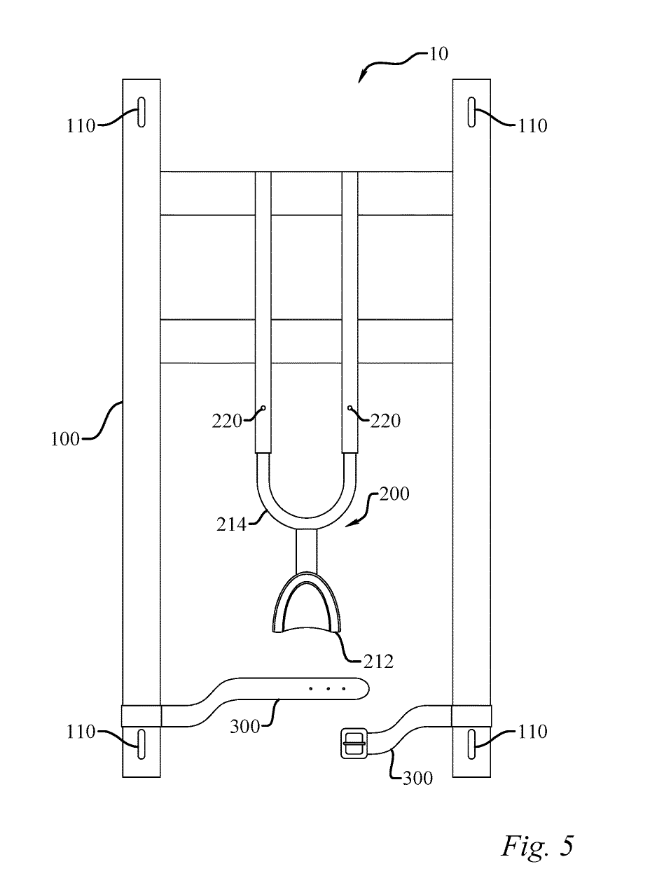

FIG. 5 shows a top view of the embodiment of FIG. 4.

These illustrations are provided to assist in the understanding of the exemplary embodiments of a cervical traction device and a method of using the same, as described in more detail below, and should not be construed as unduly limiting the specification. In particular, the relative spacing, positioning, sizing and dimensions of the various elements illustrated in the drawings may not be drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

As seen in FIGS. 1-5, certain embodiments of a cervical traction device may be seen. In one such embodiment, seen well in FIGS. 1-3, a cervical traction device (10) may include a frame (100) and two non-traumatic and releasable skull attachments (200, 300). A first skull attachment (200) may be attached to the frame (100) where it would be and releasably attachable to a skull at an anterior portion of a head on an inferior aspect of a maxilla of a user. Thus upward force would be exerted on the maxilla, and not the mandible as is common in the prior art. There may be a second skull attachment (300) attached to the frame (100) where it would be releasably attachable to the skull at a posterior portion of the head on an inferior aspect of an occiput of the user. Thus, the total upwards traction force could be distributed between the lower back of the head and the maxilla.

The skull attachments (200, 300) may be fixed to the frame, or they may be releasably attached, and may exist as a plurality of attachments to vary the positon of the skull attachments (200, 300) relative to the frame (100). As seen well in FIG. 2, the frame (100) may be provided with a plurality of suspension points (110) releasably attachable to a device external to the frame (100). In one embodiment, the plurality of suspension points (110) are arranged in an anterior-posterior direction relative to a transverse process of a cervical vertebra of the user. The use of different attachment points (110) allows the angle at which traction may be applied to the cervical vertebrae to be adjusted and varied, since the device external to the frame (100) may be a source of external traction.

As also seen well in FIGS. 2 and 3, in some embodiments, the first reversible skull attachment (200) may be a maxillary tray (210) releasably supporting the maxilla of the user. The tray (210) may fit over the teeth, or for edentulous users or those with few teeth, the tray (210) may be formed to fit directly against the soft palate over the upper gum. The maxillary tray (210) may be divided into two portions, a maxillary contact portion (212) that contacts the maxilla and a tray-frame attachment (214) that serves to attach the tray (210) to the frame (100). The tray (210) may have a tooth guard on a superior surface of the maxillary contact portion (212). In certain embodiments, the tooth guard may molded to an individual maxillary dentition of a unique user, or similarly, may be molded to the gums and/or soft palate of a user with few or no teeth.

The tray-frame attachment (214) may be releasably attached to the frame (100) by means of maxillary tray release fixtures (220) at a maxillary tray receiver (120) on the frame (100), as seen well in both FIGS. 2 and 3. These release fixtures (220) may be button releases, pins, removable bolts, or any means as would be known to one skilled in the art to releasably yet dependably join the two parts together. As seen in FIGS. 1 and 2, the second reversible skull attachment (300) may be an occipital strap (310), and that occipital strap may be adjustable both in length and with regard to its position of attachment to the frame (100). Overall, as can be seen in FIGS. 1-5, the device (10) may be intended so that the frame (100), and hence any traction force applied, does not contact a mandible of a user while in use. However, again, one skilled in the art will realize that incidental contact with the mandible, or even a certain level of intentional applied force to the mandible, may be present in some embodiments.

Various arrangements are possible between the maxillary tray (210) and the frame (100). The maxillary tray (200) may be fixed to the frame (100) or may be releasably coupled to the frame (100). A distance between the maxillary contact portion (212) and the frame (100) is adjustably variable, and may assist in adjusting the device (10) for users of different head sizes.

Various variations are likewise possible in the construction of the frame, as would be known to one skilled in the art, and they are intended as part of this specification. As seen well in FIGS. 4 and 5, the frame (100) may be formed of two or more reversibly coupled sections, and may exist in a geometric shape that is substantially planar. Such a planar shape would essentially render the frame flat, and would minimize the storage space required by the frame (100) and make it more efficient for travel and transport.

In one particular embodiment, a cervical traction device (10) may include a frame (100) having a plurality of suspension points (110), shaped to at least partially surround a head of a human user and able to releasably and reversibly surround, at least in part, a head of a human user. For the purposes of this teaching, the term “surround” is meant to include partially surround, and does not require a complete enclosure.

There may be a maxillary tray (200) attached to the frame (100) and releasably attachable to a skull at an anterior portion of a head on an inferior aspect of a maxilla of the user, and as another attachment point, there may be an occipital strap (310) attached to the frame (100) and releasably attachable to the skull at a posterior portion of the head on an inferior aspect of an occiput of the user. Thus, traction force applied to the frame (100) is borne by the maxilla and the back of the head.

In another embodiment, a cervical traction device (10) for a human user may include a frame (100) having a plurality of suspension points (110) releasably attachable to an external device to the frame (100). These suspension points (110) may be arranged in an anterior-posterior direction relative to a transverse process of a cervical vertebra of a user, that is, at relatively a right angle to an imaginary line drawn between the transverse processes of the user.

Such an embodiment could also include a maxillary tray (200) having a maxillary contact portion (212) releasably supporting a maxilla at an anterior portion of a head on an inferior aspect of a maxilla of a user. A tray-frame attachment (214) could couple the tray (210) to the frame (100). To complete the support of the head, an occipital strap (310) may be attached to the frame (100) and the strap (310) may releasably support the head at a posterior portion of the head on an inferior aspect of an occiput, or, in other words, the lower aspect of the back of the head of a user.

As to use, any of the many well-known means of supplying traction to the device (10) may employed, such as suspension or cable-weight systems, all of which would be known to one skilled in the art.

I claim:

1. A cervical traction device (10) comprising:
a frame (100);
a first skull attachment (200) attached to the frame (100) and releasably attachable to a skull at an anterior portion of a head on an inferior aspect of a maxilla of a user, and
a second skull attachment (300) attached to the frame (100) and releasably attachable to the skull at a posterior portion of the head on an inferior aspect of an occiput of the user.

2. The device according to claim 1 wherein the first skull attachment (200) is releasably attached to the frame (100).

3. The device according to claim 1 wherein the second skull attachment (300) is releasably attached to the frame (100).

4. The device according to claim 1, wherein the frame (100) is provided with a plurality of suspension points (110) releasably attachable to a device external to the frame (100).

5. The device according to claim 4, wherein the plurality of suspension points (110) are arranged in an anterior-posterior direction relative to a transverse process of a cervical vertebra of the user.

6. The device according to claim 4, wherein the device external to the frame (100) is a source of external traction.

7. The device according to claim 1, wherein the first reversible skull attachment (200) further comprises a maxillary tray (210) releasably supporting the maxilla of the user.

8. The device according to claim 7, wherein the maxillary tray (210) further comprises a maxillary contact portion (212) and a tray-frame attachment (214).

9. The device according to claim 8, wherein the maxillary contact portion (212) further comprises a tooth guard on a superior surface of the maxillary contact portion (212).

10. The device according to claim 9, wherein the tooth guard is molded to an individual maxillary dentition of a unique user.

11. The device according to claim 8, wherein the tray-frame attachment (214) is releasably attached to the frame (100) by means of maxillary tray release fixtures (220) at a maxillary tray receiver (120) on the frame (100).

12. The device according to claim 1, wherein the second reversible skull attachment (300) further comprises an occipital strap (310).

13. The device according to claim 12, wherein the occipital strap is adjustable both in length and positionally in attachment to the frame (100).

14. The device according to claim 1, wherein the frame (100) does not contact a mandible of a user while in use.

15. The device according to claim 7, wherein the maxillary tray (200) is releasably coupled to the frame (100).

16. The device according to claim 8, wherein a distance between the maxillary contact portion (212) and the frame (100) is adjustably variable.

17. The device according to claim 1, wherein the frame (100) comprises two or more reversibly coupleable sections.

18. The device according to claim 1, wherein the frame (100) is substantially planar.

19. A cervical traction device (10) comprising:
a frame (100) having a plurality of suspension points (110) releasably reversibly surrounding a head of a human user;
a maxillary tray (200) attached to the frame (100) and releasably attachable to a skull at an anterior portion of a head on an inferior aspect of a maxilla of the user, and
an occipital strap (310) attached to the frame (100) and releasably attachable to the skull at a posterior portion of the head on an inferior aspect on an occiput of the user.

20. A cervical traction device (10) for a human user comprising:
a frame (100) having a plurality of suspension points (110) releasably attachable to an external device to the frame (100) and arranged in an anterior-posterior direction relative to a transverse process of a cervical vertebra of a user;
a maxillary tray (200) having a maxillary contact portion (212) releasably supporting a maxilla at an anterior portion of a head on an inferior aspect of a maxilla of a user and a tray-frame attachment (214) coupled to the frame (100), and
an occipital strap (310) coupled to the frame (100) and releasably supporting a skull at a posterior portion of the head on an inferior aspect of an occiput.
